SEQUENCE LISTING (20 sequences)

SEQ ID NO: 1 - protein, 285 aa, Chlamydia trachomatis
MTTPDNNTIDVSFPTFVRLNVATTDLADGNKSNAVTITETATANYVNVTQDLTSSTAKLECTQDLIAQGKLIVTNPKSDISFGGRVNLADNTVNYSNGGAEVSFTNINSRQGKQYVPYGLYKNGEPKISMRSALSGGHVGSGDTGGWGAEVLWDAYTEQLKDMTDGAVTLNSSNRGKLSFTASPEAPVLFRLSVFMRKNGDWLDNGVGGRVMLYVNTTDSAGKTVRRLLGIAVCLGSTWYTTVPMFWCAATYYATSSGFFQLIVGERNFRVSSLSWSVVRLPVVP

SEQ ID NO: 2 - protein, 20 aa, Chlamydia trachomatis
AQGKLIVTNPKSDISFGGRV

SEQ ID NO: 3 - protein, 20 aa, Chlamydia trachomatis
KSDISFGGRVNLADNTVNYS

SEQ ID NO: 4 - protein, 173 aa, Chlamydia trachomatis
MKKFLLLSLMSLSSLPTFAANSTGTIGIVNLRRCLEESALGKKESAEFEKMKNQFSNSMGKMEEELSSIYSKLQDDDYMEGLSETAAAELRKKFEDLSAEYNTAQGQYYQILNQSNLKRMQKIMEEVKKASETVRIQEGLSVLLNEDIVLSIDSSADKTDAVIKVLDDSFQNN

SEQ ID NO: 5 - protein, 9 aa, Chlamydia trachomatis
YQILNQSNL

SEQ ID NO: 6 - protein, 9 aa, Chlamydia trachomatis
LNQSNLKRM

SEQ ID NO: 7 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 8 - protein, 9 aa, Chlamydia trachomatis
FSVTNPVVF

SEQ ID NO: 9 - protein, 8 aa, Artificial Sequence
  Synthetic Construct
SIINFEKL

SEQ ID NO: 10 - protein, 20 aa, Chlamydia trachomatis
NVTQDLTSSTAKLECTQDLI

SEQ ID NO: 11 - protein, 20 aa, Chlamydia trachomatis
AKLECTQDLIAQGKLIVTNP

SEQ ID NO: 12 - protein, 9 aa, Chlamydia trachomatis
SNLKRMQKI

SEQ ID NO: 13 - protein, 9 aa, Chlamydia trachomatis
AALYSTEDL

SEQ ID NO: 14 - protein, 9 aa, Chlamydia trachomatis
FQEKDADTL

SEQ ID NO: 15 - protein, 9 aa, Chlamydia trachomatis
QSVNELVYV

SEQ ID NO: 16 - protein, 9 aa, Chlamydia trachomatis
LEFASCSSL

SEQ ID NO: 17 - protein, 9 aa, Chlamydia trachomatis
FTSSNLDSP

SEQ ID NO: 18 - protein, 9 aa, Chlamydia trachomatis
SQAEGQYRL

SEQ ID NO: 19 - protein, 9 aa, Chlamydia trachomatis
GQSVNELVY

SEQ ID NO: 20 - protein, 9 aa, Chlamydia trachomatis
QAVLLLDQI

(19) United States
(12) Patent Application Publication
(10) Pub. No.: US 2010/0260791 A1
(43) Pub. Date: Oct. 14, 2010

(54) CHLAMYDIA ANTIGENS

(75) Inventors: Darren E. Higgins, Jamaica Plain, MA (US); Todd Gierahn, Brookline, MA (US); Michael N. Starnbach, Needham, MA (US); Nadia R. Roan, San Francisco, CA (US)

Correspondence Address: CLARK & ELBING LLP, 101 FEDERAL STREET, BOSTON, MA, 02110, US

(73) Assignee: President and Fellows of Harvard, Cambridge, MA (US)

(21) Appl. No.: 12/671,250

(22) PCT Filed: Aug. 1, 2008
(86) PCT No.: PCT/US08/09282

§ 371 Date: May 7, 2010

Related U.S. Application Data
(60) Provisional application No. 60/963,215, filed on Aug. 3, 2007.

(51) Int. Cl. A61K 39/118; C07K 14/195; C07K 7/08; A61K 38/16; A61K 38/08; A61P 37/04

(52) U.S. Cl. 424/190.1; 530/326; 530/350; 530/327; 514/21.2; 530/328; 530/329; 514/21.6; 424/192.1; 424/263.1

(57) ABSTRACT

Chlamydia antigens (e.g., polypeptides, polypeptide fragments, and fusion proteins) are provided. Also provided are vaccines and pharmaceutical compositions for treating or preventing a bacterial infection, such as Chlamydia, in a subject.

### STATEMENT AS TO FEDERALLY FUNDED RESEARCH

This invention was made with Government support under grant AI039558 awarded by the National Institutes of Health. The Government has certain rights to this invention.

### BACKGROUND OF THE INVENTION

Chlamydia trachomatis is an intracellular bacterial pathogen that colonizes and infects oculogenital surfaces. Ocular infections of Chlamydia trachomatis cause trachoma, a chronic follicular conjunctivitis that results in scarring and blindness. The World Health Organization (WHO) estimates that 300-500 million people worldwide are afflicted by trachoma (Resnikoff et al., Bull. WHO 82:844-851, 2004), making it the most prevalent form of infectious preventable blindness (Whitcher et al. Bull. WHO 79:214-221, 2001). Urogenital infections are the leading cause of bacterial sexually transmitted diseases (Division of STD Prevention, Sexually Transmitted Disease Surveillance 1997, Centers Dis. Cont. Prev., Atlanta, 1998) in both developing and industrialized nations (WHO, Global Prevalence and Incidence of Selected Curable Sexually Transmitted Infections: Overview and Estimates, WHO, Geneva, 2001). Moreover, sexually transmitted diseases are risk factors for the transmission of HIV (Plummer et al., J. Infect. Dis. 163:233-239, 1991), infertility (Westrom et al., Sex. Trans. Dis. 19:185-192, 1991), and human papilloma virus-induced cervical neoplasia (Anttila et al., J. Am. Med. Assoc. 285:47-51, 2001).

For all the above reasons, control of C. trachomatis infections is an important public health goal.

### SUMMARY OF THE INVENTION

The present invention features C. trachomatis antigens, and the therapeutic uses of such antigens. The antigens of the present invention may be used to treat or prevent Chlamydia infection in a subject.

In a first aspect, the present invention provides an isolated CT144 polypeptide containing a sequence substantially identical SEQ ID NO: 1, or fragment thereof, which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay (e.g., a peptide which elicits the lowest measurable value of IFN-γ in the same assay). Desirable CT144 fragments have at least 7 amino acids and/or elicit a CD4⁺ T cell response.

One preferred embodiment of the present invention is an isolated fragment of a CT144 polypeptide, which (1) includes the sequence of amino acids 67-86 (AQGKLIVTNPKSDISFGGRV; SEQ ID NO: 2) or amino acids 77-96 (KSDISFGGRVNLADNTVNYS; SEQ ID NO: 3) of the CT144 polypeptide, (2) has at least one flanking amino acid at the N- and/or C-terminus of the SEQ ID NO: 2 or 3, (3) is fewer than 280, 270, 260, 250, 240, 230, 220, 210, 200, 190, 180, 170, 160, 150, 140, 130, 120, 110, 100, 90, 80, 70, 60, 50, 40, 35, 30, 25, or 20 amino acids in length, and (4) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A related embodiment of the invention is an isolated fragment of a CT144 polypeptide, which (1) includes the sequence of SEQ ID NO: 2 or 3; (2) has at least one flanking amino acid at the N- and/or C-terminus of the SEQ ID NO: 2 or 3 sequence; (3) is fewer than 280 amino acids in length; (4) contains one or more, preferably 1, 2, 3, 4, 5, 6, 7, 8, 9, or 10 conservative amino acid substitutions in the sequence of SEQ ID NO: 2 or 3; and (5) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

One preferred embodiment is an isolated fragment of a CT144 polypeptide having the sequence of SEQ ID NO: 2 or 3 which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A further embodiment of the invention is an isolated fragment of a CT144 polypeptide, having of the sequence of SEQ ID NO: 2 or 3, that is truncated by 1, 2, 3, 4, 5, or 6 amino acids at the N- and/or C-terminus of the polypeptide, and which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

Another embodiment of the invention, is an isolated fragment of a CT144 polypeptide (1) consisting of the sequence of SEQ ID NO: 2 or 3, (2) containing one or more, preferably 1, 2, 3, 4, 5, 6, 7, 8, 9, or 10 conservative amino acid substitutions, and (3) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

The present invention further provides an isolated fragment of a CT242 polypeptide (SEQ ID NO: 4), which (1) includes amino acids 109-117 (YQILNQSNL; SEQ ID NO: 5) or amino acids 112-120 (LNQSNLKRM; SEQ ID NO: 6) of the CT242 polypeptide; (2) has fewer than 170, 160, 150, 140, 130, 120, 110, 100, 90, 80, 70, 60, 50, 40, 30, 25, 20, 15, or 10 amino acids; and (3) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay. Desirable CT242 fragments have at least 7 amino acids and/or elicit a CD8⁺ T cell response.

One preferred embodiment of the present invention is an isolated fragment of a CT242 polypeptide: (1) containing the sequence of SEQ ID NO: 5 or 6; (2) having and at least one flanking amino acid at the N- and/or C-terminus of the SEQ ID NO: 5 or 6 sequence; (3) is fewer than 170 amino acids in length; and (4) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A related embodiment of the invention is an isolated fragment of a CT242 polypeptide, which (1) includes the sequence of SEQ ID NO: 5 or 6; (2) has at least one flanking amino acid at the N- and/or C-terminus of the SEQ ID NO: 5 or 6 sequence; (3) is fewer than 170 amino acids in length; (4) contains one or more, preferably 1, 2, 3, 4, or 5 conservative amino acid substitutions in the sequence of SEQ ID NO: 7, 8, or 9; and (4) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

One preferred embodiment is an isolated fragment of a CT242 polypeptide having the sequence of SEQ ID NO: 5 or 6, which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A further embodiment of the invention is an isolated fragment of a CT242 polypeptide having of the sequence of SEQ ID NO: 5 or 6, that is truncated by one or two amino acids at the N- and/or C-terminus of the polypeptide, which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

Another embodiment of the invention, is an isolated fragment of a CT242 polypeptide (1) having the sequence of SEQ ID NO: 5 or 6, (2) containing one or more, preferably 1, 2, 3, 4, or 5 conservative amino acid substitutions, which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

The present invention further provides an isolated fragment of a CT812 polypeptide (SEQ ID NO: 7), which (1) includes amino acids 103-111 (FSVTNPVVF; SEQ ID NO: 8) of the CT812 polypeptide, (2) has fewer than 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 25, 20, 15, or 10 amino acids, and (3) elicits which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay. Desirably, a CT812 fragment has at least seven amino acids and/or elicits a CD8⁺ T-cell response.

One preferred embodiment of the present invention is an isolated fragment of a CT812 polypeptide: (1) containing the sequence of SEQ ID NO: 8; (2) having and at least one flanking amino acid at the N- and/or C-terminus of the SEQ ID NO: 8 sequence; (3) is fewer than 770 amino acids in length; and (4) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A related embodiment of the invention is an isolated fragment of a CT812 polypeptide, which (1) includes the sequence of SEQ ID NO: 8; (2) has at least one flanking amino acid at the N- and/or C-terminus of the SEQ ID NO: 8 sequence; (3) is fewer than 770 amino acids in length; (4) contains one or more, preferably 1, 2, 3, 4, or 5 conservative amino acid substitutions in the sequence of SEQ ID NO: 8; and (5) elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

One preferred embodiment is an isolated fragment of a CT812 polypeptide having the sequence of SEQ ID NO: 8 which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A further embodiment of the invention is an isolated fragment of a CT812 polypeptide, having the sequence of SEQ ID NO: 8, that is truncated by one or two amino acids at the N- and/or C-terminus of the polypeptide, which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

Another embodiment of the invention, is an isolated fragment of a CT812 polypeptide having the sequence of SEQ ID NO: 8, containing one or more, preferably 1, 2, 3, 4, or 5 conservative amino acid substitutions, which elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production elicited from a non-immunogenic peptide in the same assay.

A further aspect of the invention is an isolated fusion protein containing (1) the sequence of any of above CT144 polypeptides or fragments of the invention, and (2) a fusion partner.

The present invention further provides a fusion protein having (1) the sequence of any of the above CT242 polypeptides or fragments of the invention, and (2) a fusion partner.

The present invention further features an isolated fusion protein having (1) the sequence of any of the above the CT812 polypeptide or fragments of the invention, and (2) a fusion partner.

The invention further provides pharmaceutical compositions containing any of the above described polypeptides, fragments, and fusion proteins of the invention and a pharmaceutically acceptable carrier.

The invention additionally provides vaccines containing any of the above described polypeptides, fragments, and fusion proteins of the invention and a pharmaceutically acceptable carrier. Additionally, the invention provides DNA vaccines containing a polynucleotide sequence that encodes any of the above described polypeptides, fragments, and fusion proteins of the invention and a pharmaceutically acceptable carrier.

In preferred embodiments of all the above aspects, the polypeptides, polypeptide fragments, fusion proteins, and vaccines of the invention (e.g., protein and DNA vaccines) elicit an immune response when administered to a mammal. Desirably, the polypeptides, polypeptide fragments, fusion proteins, and vaccines of the invention elicit an immune response when administered to a human.

The invention further provides a method of treating or preventing a bacterial infection, preferably a Chlamydia infection, by administering to a subject in need thereof (e.g., a subject who has or is at risk for contracting Chlamydia), a therapeutically effective amount of any of the above described polypeptides, fragments, fusion proteins, vaccines (e.g., protein vaccines or DNA vaccines) of the present invention. In desirable embodiments of the method, the polypeptide, fragment, fusion protein, or vaccine (e.g., protein vaccines or DNA vaccines) of the present invention is capable of generating an immune response in a subject and/or is administered in a pharmaceutically acceptable carrier.

### DEFINITIONS

By a “CT144 polypeptide” is meant a polypeptide that is substantially identical to the amino acid sequence of SEQ ID NO: 1. Desirably, a CT144 polypeptide has at least 80%, 85%, 90%, 95%, 99%, or even 100% sequence identity to the amino acid sequence of SEQ ID NO: 1. Desirably, a CT144 polypeptide elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay).

By a “fragment of a CT144 polypeptide” or a “CT144 fragment” is meant a fragment of a CT144 polypeptide that contains fewer than 280 amino acids. Desirably, a CT144 fragment elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay). Desirably, the fragment contains fewer than 270, 260, 250, 240, 230, 220, 210, 200, 190, 180, 170, 160, 150, 140, 130, 120, 110, 100, 90, 80, 70, 60, 50, 40, 35, 30, 25, or 20 amino acids, and desirably, is immunogenic. Desirably, a CT144 fragment contains the sequence of SEQ ID NO: 2 or 3, and has fewer than 280 amino acids. Preferred CT144 fragments are between 7 and 279 amino acids in length (e.g., 7, 8, 9, 10, 11, 12, 13, 14, 15, 16, 17, 18, 19, 20, 50, 100, or 150 amino acids in length). A CT144 fragment may contain one or more conservative amino acid substitutions in the sequence of SEQ ID NO: 2 or 3. Additional desirable CT144 fragments consist of the sequence of SEQ ID NO: 2 or 3, or contain one or more conservative amino acid substitutions in the sequence of SEQ ID NO: 2 or 3, and/or at least one flanking amino acid at the N- and/or C-terminus of the sequence of SEQ ID NO: 2 or 3. Other preferred CT 144 fragments contain seven or more continuous amino acids of the sequence of SEQ ID NO: 2 or 3.

By a “CT242 polypeptide” is meant a polypeptide that is substantially identical to the amino acid sequence of SEQ ID NO: 4. Desirably, a CT242 polypeptide has at least 80%, 85%, 90%, 95%, 99%, or even 100% sequence identity to the amino acid sequence of SEQ ID NO: 4. Desirably, a CT242 polypeptide elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay).

By a “fragment of a CT242 polypeptide” or “CT242 fragment” is meant a fragment of a CT242 polypeptide containing fewer than 170 amino acids. Desirably, a CT242 fragment elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay). Desirably, the fragment is fewer than 160, 150, 140, 130, 120, 110, 100, 90, 80, 70, 60, 50, 40, 30, 25, 20, 15, or 10 amino acids in length, and desirably, is immunogenic. Preferred CT242 fragments are between 7 and 169 amino acids in length (e.g., 7, 8, 9, 10, 11, 12, 13, 14, 15, 16, 17, 18, 19, 20, 50, 100, or 150 amino acids in length). Desirably, a CT242 fragment contains the sequence of SEQ ID NO: 5 or 6, and has fewer than 170 amino acids. A CT242 fragment may contain one or more conservative amino acid substitutions in the sequence of SEQ ID NO: 5 or 6. Additional desirable CT242 fragments consist of the sequence of SEQ ID NO: 5 or 6, or contain one or more conservative amino acid substitutions in the sequence of SEQ ID NO: 5 or 6, and/or at least one flanking amino acid at the N- and/or C-terminus of the sequence of SEQ ID NO: 5 or 6. Other preferred CT242 fragments contain seven or more continuous amino acids of the sequence of SEQ ID NO: 5 or 6.

By a “CT812 polypeptide” is meant a polypeptide that is substantially identical to the amino acid sequence of SEQ ID NO: 7. Desirably, a CT812 polypeptide has at least 80%, 85%, 90%, 95%, 99%, or even 100% identity to the amino acid sequence of SEQ ID NO: 7. Desirably, a CT812 polypeptide elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay).

By “fragment of a CT812 polypeptide” or a “CT812 fragment” is meant a fragment of a CT812 polypeptide containing fewer than 770 amino acids. Preferred CT812 fragments are between 7 and 769 amino acids in length (e.g., 7, 8, 9, 10, 11, 12, 13, 14, 15, 16, 17, 18, 19, 20, 50, 100, or 150 amino acids in length). Desirably, a CT812 fragment elicits at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay). Desirably, the fragment is fewer than 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 25, 20, 15, or 10 amino acids, and desirably, is immunogenic. Desirably, a CT812 fragment contains the sequence of SEQ ID NO: 8, and has fewer than 770 amino acids. A CT812 fragment may contain one or more conservative amino acid substitutions in the sequence of SEQ ID NO: 8. Additional desirable CT812 fragments consist of the sequence of SEQ ID NO: 8, or contain one or more conservative amino acid substitutions in the sequence of SEQ ID NO: 8 and/or at least one flanking amino acid at the N- and/or C-terminus of the sequence of SEQ ID NO: 8. Other preferred CT812 fragments contain seven or more continuous amino acids of the sequence of SEQ ID NO: 8.

By “substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Multiple sequences may also be aligned using the Clustal W(1.4) program (produced by Julie D. Thompson and Toby Gibson of the European Molecular Biology Laboratory, Germany and Desmond Higgins of European Bioinformatics Institute, Cambridge, UK) by setting the pairwise alignment mode to “slow,” the pairwise alignment parameters to include an open gap penalty of 10.0 and an extend gap penalty of 0.1, as well as setting the similarity matrix to “blosum.” In addition, the multiple alignment parameters may include an open gap penalty of 10.0, an extend gap penalty of 0.1, as well as setting the similarity matrix to “blosum,” the delay divergent to 40%, and the gap distance to 8.

By “conservative amino acid substitution,” as used herein, is meant replacement, in an amino acid sequence, of an amino acid for another within a family of amino acids that are related in the chemical nature of their side chains. Genetically encoded amino acids can be divided into four families: acidic (aspartate, glutamate); basic (lysine, arginine, histidine); nonpolar (alanine, valine, leucine, isoleucine, proline, phenylalanine, methionine, tryptophan); and uncharged polar (glycine, asparagine, glutamine, cysteine, serine, threonine, tyrosine). Phenylalanine, tryptophan, and tyrosine are sometimes grouped as aromatic amino acids. In similar fashion, the amino acids can also be separated into the following groups: acidic (aspartate, glutamate); basic (lysine, arginine, histidine); alipathic (glycine, alanine, valine, leucine, isoleucine, serine, threonine), with serine and threonine optionally grouped separately as alipathic-hydroxyl; aromatic (phenylalanine, tyrosine, tryptophan); amide (asparagine, glutamine); and sulfur-containing (cysteine, methionine).

Whether a change in the amino acid sequence results in a functional homolog can be determined by assessing the ability of the variant peptide to function in a fashion similar to the wild-type protein using standard methods such as the assays described herein. For example, C. trachomatis-specific CD4⁺ or CD8⁺ cells may be used to determine whether specific C. trachomatis polypeptides or fragments thereof, are immunogenic. Desirable embodiments of the invention, include at least one conservative amino acid substitution in the amino acid sequence of SEQ ID NO: 2, 3, 5, 6, or 8; and more desirably 1, 2, 3, 4, 5, 6, 7, 8, 9, or 10 conservative amino acid substitutions in the sequence of SEQ ID NO: 2 or 3; and 1, 2, 3, 4, or 5 conservative amino acid substitutions in the sequence of SEQ ID NO: 5, 6, or 8.

By “flanking amino acid” is meant an amino acid in a polypeptide sequence that is immediately adjacent to the N- or C-terminus of a particular defined sequence. Desirably, a flanking amino acid is present on the N- and/or C-terminus of the amino acid sequence of SEQ ID NO: 2, 3, 5, 6, or 8. For the sequence of SEQ ID NO: 2 or 3, the flanking amino acids may consist of one or more naturally adjoining amino acids present in the sequence of SEQ ID NO: 1. For the sequence of SEQ ID NO: 5 or 6, the flanking amino acids may consist of one or more naturally adjoining amino acids present in the sequence of SEQ ID NO: 4. For the sequence of SEQ ID NO: 8, the flanking amino acids may consist of one or more naturally adjoining amino acids present in the sequence of SEQ ID NO: 7.

As used herein “fusion protein” refers to a polypeptide consisting of (1) a fragment of a CT144 polypeptide, fragment of a CT242 polypeptide, or fragment of a CT812 polypeptide; and (2) a fusion partner.

As used herein “fusion partner” refers to a heterologous sequence that can be fused to a fragment of a CT144 polypeptide, fragment of a CT242 polypeptide, or fragment of a CT812 polypeptide of the present invention. Desirably, the fusion partner provides a new function or activity to the fragment of a CT144 polypeptide, the fragment of a CT242 polypeptide, or the fragment of a CT812 polypeptide. Examples of fusion partners are described herein and include detection markers, DNA binding domains, gene activation domains, stabilizing domains, or sequences which aid in production or purification of the protein.

As used herein “immune response” refers to the activation of an organism's immune system in response to an antigen or infectious agent. In vertebrates, this may include, but is not limited to, one or more of the following: naïve B cell maturation into memory B cells; antibody production by plasma cells (effector B cells); induction of cell-mediated immunity; activation and cytokine release by CD4⁺ T cells; activation and cytokine release of CD8⁺ T cells; cytokine recruitment and activation of phagocytic cells (e.g., macrophages, neutrophils, eosinophils); and/or complement activation.

By “immunogenic” is meant any substance that is capable of inducing an immune response in a subject.

By “non-antigenic” is meant any peptide which elicits the lowest level of interferon-γ production compared to other tested peptides in the T-lymphocyte assays described in the Examples. The non-antigenic peptide may be a human peptide or a Chlamydia trachomatis peptide.

By “pharmaceutically acceptable salt” is meant any non-toxic acid addition salt or metal complex used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid, phosphoric acid, or the like. Metal complexes include zinc, iron, and the like.

By “pharmaceutically acceptable carrier” is meant any solution used to solubilize and deliver an agent to a subject. A desirable pharmaceutically acceptable carrier is saline. In desirable embodiments, a pharmaceutically acceptable carrier includes an adjuvant. Exemplary adjuvants are described herein. Other physiologically acceptable carriers and their formulations are known to one skilled in the art and described, for example, in Remington's Pharmaceutical Sciences, (19th edition), ed. A. Gennaro, 1995, Mack Publishing Company, Easton, Pa.

By “isolated” is meant a protein (or a fragment thereof) that has been separated from components that naturally accompany it. Typically, the polypeptide is substantially isolated when it is at least 60%, by weight, free from the proteins and naturally occurring organic molecules with which it is naturally associated. The definition also extends to a polypeptide separated from its flanking amino acids (e.g., for an amino acid sequence, isolated refers to a sequence that is free from the flanking amino acids with which the sequence is naturally associated in a polypeptide). Preferably, the polypeptide is at least 75%, more preferably at least 90%, and most preferably at least 99%, by weight, isolated. An isolated polypeptide may be obtained by standard techniques, for example, by extraction from a natural source (e.g., purification from a cell infected with C. trachomatis), by expression of a recombinant nucleic acid encoding a fragment of the CT144, CT242, or CT812 polypeptide, or by chemically synthesizing the polypeptide. Purity can be measured by any appropriate method, e.g., by column chromatography, polyacrylamide gel electrophoresis, or HPLC analysis.

By a “therapeutically effective amount” is meant the amount of a immunogenic compound (e.g., polypeptide, fragment, fusion protein, or vaccine) required to generate in a subject one or more of the following effects: an immune response; a decrease in the level of Chlamydia infection (e.g., a reduction of at least 5%, 10%, 20%, or 30%; more desirably 40%, 50%, 60%, or 70%; and most desirably 80% or 90%); or increased resistance to a new Chlamydia infection (e.g., an increase of at least 5%, 10%, 20%, 30%, 40%, or 50%; more desirably 60%, 70%, 80%, or 90%; or most desirably 100%, 200%, or 300%).

### BRIEF DESCRIPTION OF THE DRAWINGS

FIG. 1 is the complete amino acid sequence of the polypeptide CT144 (SEQ ID NO: 1) (Genbank Accession number NP_{—}219647).

FIG. 2 is the complete amino acid sequence of the polypeptide CT242 (SEQ ID NO: 4) (Genbank Accession number NP_{—}219747).

FIGS. 3-1 to 3-2 are the complete amino acid sequence of the polypeptide CT812 (SEQ ID NO: 7) (Genbank Accession number NP_{—}220332).

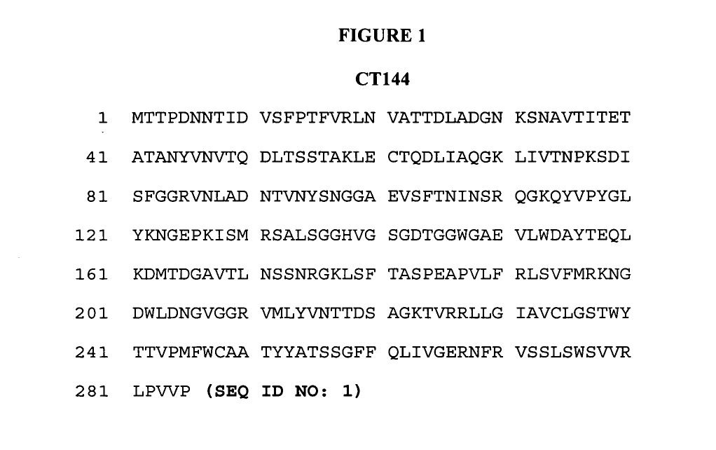

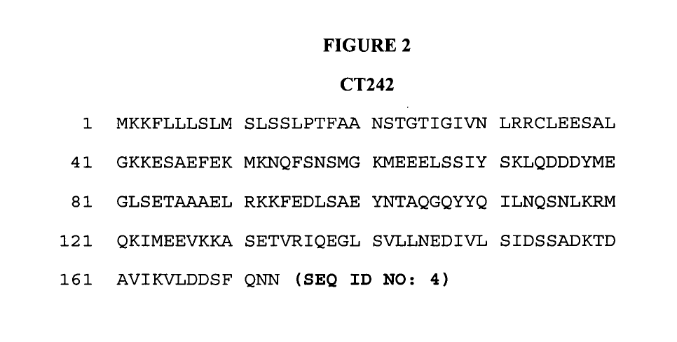

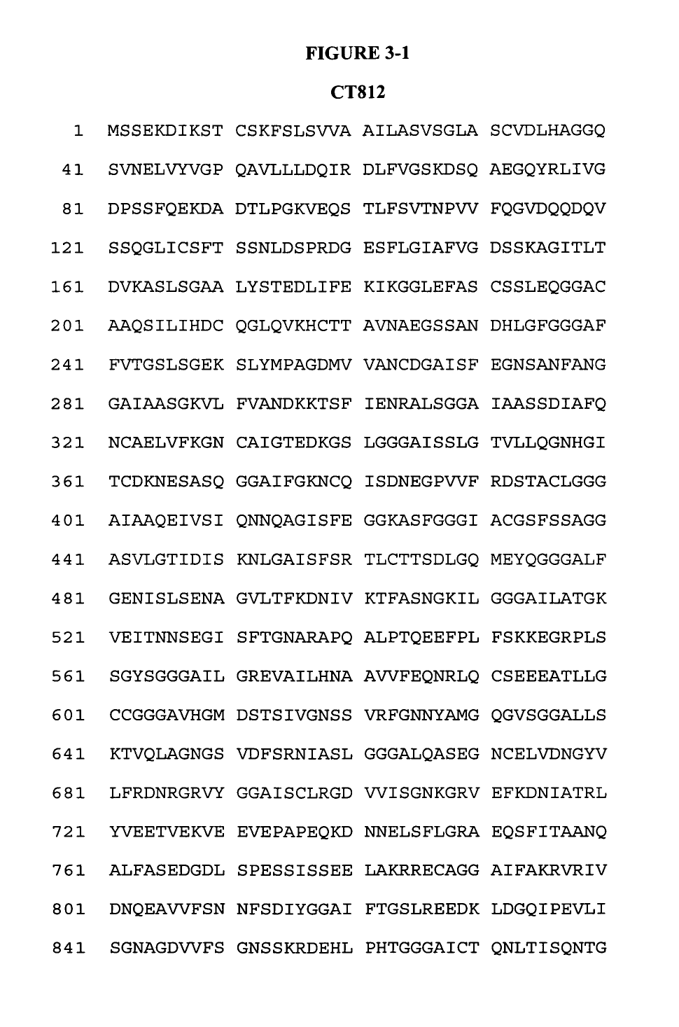

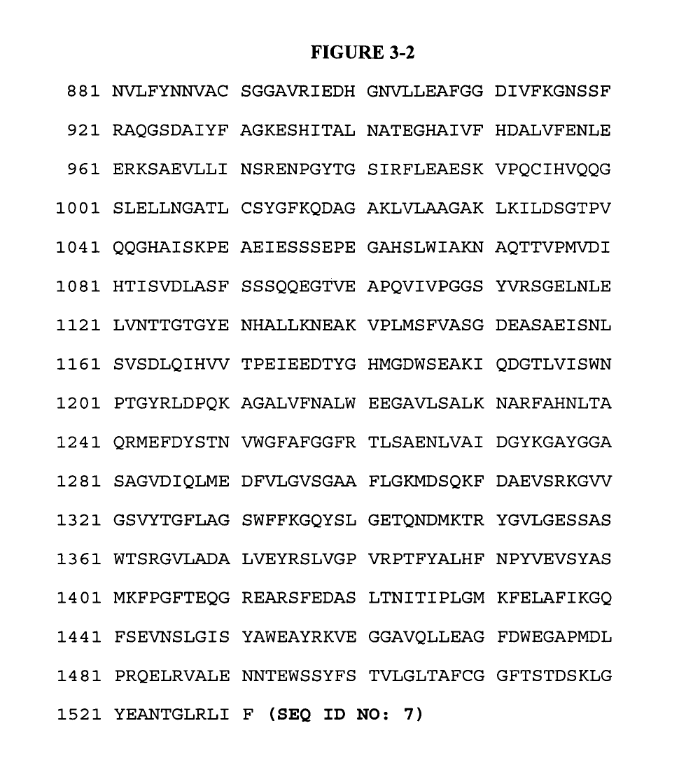

### DETAILED DESCRIPTION

Previous attempts to develop a Chlamydial vaccine have met with little success (Cotter et al., Infect. Immun. 63:4704-4714, 1995) (Pal et al., Vaccine 17:459-465, 1999) (Pal et al., Infect. Immun. 65:3361-3369, 1997) (Su et al., Vaccine 13:1023-1032, 1995) (Taylor et al., Invest. Opthalmol. Vis. Sci. 29:1847-1853, 1988) (Zhang et al., J. Infect. Dis. 176:1035-1040, 1997). Subunit vaccines have the potential to be able to control many important human pathogens which have thus far resisted classical vaccination strategies.

Chlamydia trachomatis is a human pathogen against which a protective vaccine has not been developed even though it is a significant burden on human society. It is the most common bacterial cause of sexually transmitted disease in the United States. Chronic inflammation in the female genital tract caused by C. trachomatis can lead to serious pathologies such as pelvic inflammatory disease and ectopic pregnancy. C. trachomatis is also the most common cause of preventable blindness worldwide with an estimated 1-1.5 million people currently blind from the disease.

Use of classical vaccinology methods did not yield a successful vaccine against C. trachomatis pathogen because immunization with killed bacteria leads to an increase in the severity of the pathologies associated with the disease and the lack of a genetic system to manipulate the bacterium has prevented the development of attenuated Chlamydia strains. A subunit vaccine in which specific proteins from C. trachomatis are used to elicit an immune response has the potential to overcome the barriers to a successful vaccine by eliciting responses to protective antigens while avoiding the pathological responses associated with immunization with the entire organism. To make a successful C. trachomatis subunit vaccine, the proteins in the C. trachomatis proteome that elicit protective immune responses must be identified. We report here the identification of new C. trachomatis proteins that elicit CD8⁺ and CD4⁺ T-cell responses during C. trachomatis infection.

The immunogenic Chlamydia peptides of the present invention were identified in an assay utilizing C. trachomatis-specific CD4⁺ or CD8⁺ T cells, and an expression library of genomic sequences from C. trachomatis serovar D. A detailed description of the assay and its components is provided below.

The invention features CT144, CT242, and CT812 polypeptides, polypeptide fragments, and fusion proteins. The invention further features compositions, vaccines (e.g., DNA vaccines), and kits containing a CT144, CT242, or CT812 polypeptide, polypeptide fragment, or fusion protein (or a polynucleotide sequence encoding a polypeptide, polypeptide fragment, or fusion protein of the present invention).

Methods for the addition of flanking amino acids to the amino or carboxy ends of a specific protein sequence are well known in the art. The flanking amino acids added may be the naturally adjoining sequences present in the full-length sequence of the naturally-occurring polypeptide (e.g., for a CT144 fragment, the adjoining sequence in the sequence of SEQ ID NO: 1; for a CT242 fragment, the adjoining sequence in the sequence of SEQ ID NO: 4; and for a CT812 fragment, the adjoining sequence in the sequence of SEQ ID NO: 7), or may comprise any other amino acid sequence.

In addition, the invention also provides fusion proteins consisting of (1) any of the CT144, CT242, or CT812 polypeptides or polypeptide fragments of the present invention, and (2) a fusion partner. A fusion partner is a heterologous protein sequence that may provide an additional function or activity to the fragment of the invention. For example, a fusion partner may be detected directly or indirectly (e.g., green fluorescent protein (GFP), hemagglutinin, or alkaline phosphatase), provide a DNA binding domain (e.g., GAL4 or LexA), provide a gene activation domain (e.g., GAL4 or VP16), stabilize the polypeptide, or facilitate its production or purification (e.g., His₆, a myc tag, streptavidin, a SIINFEKL epitope (SEQ ID NO: 9), or a secretion signal).

The fusion partner may also contain sequences which provide immunostimulatory function, examples include interleukin-2 (Fan et al., Acta Biochim. Biophys. Sin. 38:683-690, 2006), immunoglobulin (e.g., IgG, IgM, IgE, or IgA), Toll-like receptor-5 flagellin (Huleatt et al., Vaccine 8:763-775, 2007), simian immunodeficiency virus Tat (Chen et al., Vaccine 24:708-715, 2006), or fibrinogen-albumin-IgG receptor of group C streptococci (Schulze et al., Vaccine 23:1408-1413, 2005). In addition, fusion partner sequences may be added to enhance solubility or increase half-life, for example, hydrophilic amino acid residues (Murby et al., Eur. J. Biochem. 230:38-44, 1995), glycosylation sequences (Sinclair and Elliott, J. Pharm. Sci. 94:1626-1635, 2005), or the carboxy terminus of human chorionic gonadotropin or thrombopoeitin (Lee et al., Biochem. Biophys. Res. Comm. 339:380-385, 2006). Methods for the addition of these flanking sequences are known in the art and further described herein.

In addition, methods for introducing conservative amino acid substitutions into a polypeptide sequence are also known in the art. Amino acids within the sequence of SEQ ID NOS: 2, 3, 5, 6, and 8 can be replaced with other amino acids having similar chemical characteristics. For example, a conservative substitution is replacing one acidic amino acid for another (e.g., aspartate for glutamate, or vice versa). Another example, is replacing one basic amino acid for another (lysine for histidine, or vice versa).

Methods for removing amino acids from the amino and/or carboxy end of a polypeptide sequence are also known in the art. Amino acids desirably are removed from the amino and/or carboxy end of the protein fragment of SEQ ID NO: 2, 3, 5, 6, or 8.

The specific polypeptides, polypeptide fragments, or fusion proteins disclosed herein can be assayed for their immunogenicity using standard methods as described, for instance, in the Example below.

### CT144, CT242, and CT812 Polypeptide, Polypeptide Fragment, or Fusion Protein Expression

The CT144, CT242, and CT812 polypeptides, polypeptide fragments, or fusion proteins of the present invention may be produced by transformation of a suitable host cell with a polynucleotide molecule encoding the polypeptide fragment or fusion protein in a suitable expression vehicle.

Those skilled in the field of molecular biology will understand that any of a wide variety of expression systems may be used to provide the CT144, CT242, and CT812 polypeptides, polypeptide fragments, or fusion proteins disclosed herein. The precise host cell used is not critical to the invention. The CT144, CT242, and CT812 polypeptides, polypeptide fragments, or fusion proteins may be produced in prokaryotic host (e.g., E. coli) or in a eukaryotic host (e.g., S. cerevisiae, insect cells, e.g., Sf21 cells, or mammalian cells, e.g., NIH 3T3, HeLa, or preferably COS cells). Such cells are available from a wide range of sources (e.g., the American Type Culture Collection, Manassas, Va.). The method of transformation or transfection and the choice of expression vehicle will depend on the host system selected. Transformation and transfection methods are described, e.g., in Kucherlapati et al. (CRC Crit. Rev. Biochem. 16:349-379, 1982) and in DNA Transfer to Cultured Cells (eds., Ravid and Freshney, Wiley-Liss, 1998); and expression vehicles may be chosen from those provided, e.g., in Vectors: Expression Systems: Essential Techniques (ed., Jones, Wiley & Sons Ltd., 1998).

Once the recombinant polypeptide, polypeptide fragment, or fusion protein is expressed, it can be isolated, e.g., using affinity chromatography. In one example, an antibody raised against a CT144, CT242, or CT812 polypeptide, polypeptide fragment, or fusion protein may be attached to a column and used to isolate the recombinant polypeptide, polypeptide fragment, or fusion protein. Lysis and fractionation of polypeptide-, polypeptide fragment-, or fusion protein-harboring cells prior to affinity chromatography may be performed by standard methods (see, e.g., Methods in Enzymology, volume 182, eds., Abelson, Simon, and Deutscher, Elsevier, 1990).

Once isolated, the recombinant CT144, CT242, and CT812 polypeptides, polypeptide fragments, or fusion proteins can, if desired, be further purified, e.g., by high performance liquid chromatography (see e.g., Fisher, Laboratory Techniques in Biochemistry and Molecular Biology, eds., Work and Burdon, Elsevier, 1980; and Scopes, Protein Purification: Principles and Practice, Third Edition, ed., Cantor, Springer, 1994).

The CT144, CT242, and CT812 polypeptides, polypeptide fragments, or fusion proteins can also be produced by chemical synthesis (e.g., by the methods described in Solid Phase Peptide Synthesis, 2nd ed., 1984, The Pierce Chemical Co., Rockford, Ill.; and Solid-Phase Synthesis: A Practical Guide, ed., Kates and Albericio, Marcel Dekker Inc., 2000).

For production of stable cell lines expressing the polypeptides described herein, PCR-amplified nucleic acids encoding any of the CT144, CT242, or CT812 polypeptides, polypeptide fragments, or fusion proteins of the present invention may be cloned into the restriction site of a derivative of a mammalian expression vector. For example, KA, which is a derivative of pcDNA3 (Invitrogen, Carlsbad, Calif.) contains a DNA fragment encoding an influenza virus hemagglutinin (HA). Alternatively, vector derivatives encoding other tags, such as c-myc or poly-histidine tags, can be used.

### Vaccine Production

The invention also provides for a vaccine composition including the CT144, CT242, or CT812 polypeptides, polypeptide fragments, or fusion proteins of the present invention. The invention also provides DNA vaccines which contain polynucleotide sequences encoding the CT144, CT242, or CT812 polypeptides, polypeptide fragments, or fusion proteins of the present invention. Preferred polypeptides, polypeptide fragments, or fusion proteins, for use in a vaccine composition elicit at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay). Likewise, preferred polynucleotide sequences for use in a DNA vaccine contain polynucleotide sequences encoding CT144, CT242, or CT812 polypeptides, polypeptide fragments, or fusion proteins of the present invention which elicit at least a 3-, 4-, 5-, 6-, 7-, 8-, 10-, 20-, 30-, 40-, 50-, 60-, 70-, 80-, 90-, 100-, 200-, or 500-fold increase in interferon-γ production from a population of T-lymphocytes compared to the level of interferon-γ production from T-lymphocytes treated with a non-antigenic peptide in the same assay (e.g., a peptide which elicits the lowest measurable value of IFN-γ in the same assay). The invention further includes a method of inducing an immunological response in a subject, particularly a human, the method including inoculating a subject with a CT144, CT242, or CT812 polypeptide, polypeptide fragment, or fusion protein disclosed herein, or a DNA vaccine containing a polynucleotide sequence encoding a CT144, CT242, or CT812 polypeptide, polypeptide fragment, or fusion protein disclosed herein, in a suitable carrier for the purpose of inducing an immune response to prevent or protect a subject from infection, desirably bacterial infection, and most desirably, C. trachomatis infection. The administration of this immunological composition (e.g., DNA vaccine) may be used either therapeutically in subjects already experiencing an infection, or may be used prophylactically to prevent an infection. In addition, the above described vaccines can also be administered to subjects to generate polyclonal antibodies (purified or isolated from serum using standard methods) that may be used to passively immunize a subject. These polyclonal antibodies can also serve as immunochemical reagents.

The preparation of vaccines that contain immunogenic polypeptides is known to one skilled in the art. The CT144, CT242, or CT812 polypeptides, polypeptide fragments, or fusion proteins of the present invention may serve as an antigen for vaccination. Both the protein-based vaccines described herein and DNA vaccines encoding the polypeptides, polypeptide fragments, or fusion proteins of the present invention may be delivered to a subject in order to induce an immunological response comprising the production of antibodies, or, in particular, a CD4⁺ and/or CD8⁺ T cell response in a subject.

Protein-based vaccines are typically prepared from a purified recombinant CT144, CT242, or CT812 polypeptide, polypeptide fragment, or fusion protein of the present invention in a physiologically acceptable diluent vehicle such as water, phosphate-buffered saline (PBS), acetate-buffered saline (ABS), Ringer's solution, or the like to form an aqueous composition. The diluent vehicle can also include oleaginous materials such as squalane, or squalene as is discussed below.

Vaccine antigens are usually combined with a pharmaceutically acceptable carrier, which includes any carrier that does not include the production of antibodies harmful to the subject receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

The CT144, CT242, and CT812 polypeptides, polypeptide fragments, or fusion proteins of the present invention may be mixed with excipients that are pharmaceutically acceptable and compatible with the immunogenic polypeptide, polypeptide fragment, or fusion protein. Suitable excipients are, for example, water, saline, dextrose, glycerol, ethanol, or the like and combinations thereof. In addition, if desired, a vaccine can contain minor amounts of auxiliary substances such as wetting or emulsifying agents, or pH buffering agents that enhance the immunogenic effectiveness of the composition.

A protein-based vaccine advantageously also includes an adjuvant. Suitable adjuvants for vaccines of the present invention comprise those adjuvants that are capable of enhancing the B cell and/or T cell response (e.g., CD4⁺ and/or CD8⁺ T cell response) to the immunogenic polypeptide or fragment of the present invention. Adjuvants are well known in the art (see, e.g., Vaccine Design—The Subunit and Adjuvant Approach, 1995, Pharmaceutical Biotechnology, Volume 6, Eds. Powell and Newman, Plenum Press, New York and London).

Preferred adjuvants for use with the immunogens of the present invention include aluminum or calcium salts (e.g., hydroxide or phosphate salts). A desirable adjuvant is an aluminum hydroxide gel such as Alhydrogel™. For aluminum hydroxide gels (alum), the immunogenic polypeptide fragment or fusion protein is admixed with the adjuvant so that between 50 to 800 μg of aluminum are present per dose, and preferably, between 400 and 600 μg are present.

Another adjuvant for use with an immunogenic polypeptide, polypeptide fragment, or fusion protein of the present invention is an emulsion. An emulsion can be an oil-in-water emulsion or a water-in-oil emulsion. In addition to the immunogenic polypeptide, polypeptide fragment, or fusion protein, such emulsions comprise an oil phase of squalene, squalane, or the like, as are well known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate, and mannide mono-oleate. An immunogen-containing emulsion is administered as an emulsion.

Desirably, such emulsions are water-in-oil emulsions that comprise squalene and mannide mono-oleate (Arlacel™ A), optionally with squalane, emulsified with the immunogenic polypeptide fragment or fusion protein in an aqueous phase. Well-known examples of such emulsions include Montanide™ ISA-720 and Montanide™ ISA-703 (Seppic, Castres, France), each of which is understood to contain both squalene and squalane, with squalene predominating in each, but to a lesser extent in Montanide™ ISA-703. Desirably, Montanide™ ISA-720 is used, and a ratio of oil-to-water of 7:3 (w/w) is used. Other preferred oil-in-water emulsion adjuvants include those disclosed in WO 95/17210 and EP 0399842, herein incorporated by reference.

The use of small molecule adjuvants is also contemplated herein. One type of small molecule adjuvant useful herein is a 7-substituted-8-oxo- or 8-sulfo-guanosine derivative described in U.S. Pat. Nos. 4,539,205; 4,643,992; 5,011,828; and 5,093,318; herein incorporated by reference. Of these materials, 7-allyl-8-oxoguanosine (loxoribine) is particularly preferred. Loxoribine has been shown to be particularly effective in inducing an immunogen-specific response.

Additional useful adjuvants include monophosphoryl lipid A (MPL) available from Corixa Corp. (see, U.S. Pat. No. 4,987,237), CPG available from Coley Pharmaceutical Group, QS21 available from Aquila Biopharmaceuticals, Inc., SBAS2 available from SmithKline Beecham, the so-called muramyl dipeptide analogues described in U.S. Pat. No. 4,767,842, and MF59 available from Chiron Corp. (see, U.S. Pat. Nos. 5,709,879 and 6,086,901). Further adjuvants include the active saponin fractions derived from the bark of the South American tree Quillaja Saponaria Molina (e.g., Quil™ A). Derivatives of Quil™ A, for example QS21 (an HPLC purified fraction derivative of Quil™ A), and the method of its production is disclosed in U.S. Pat. No. 5,057,540. In addition to QS21 (known as QA21), other fractions such as QA17 are also disclosed.

3-De-O-acylated monophosphoryl lipid A is a well-known adjuvant manufactured by Ribi Immunochem. The adjuvant contains three components extracted from bacteria: monophosphoryl lipid (MPL) A, trehalose dimycolate (TDM), and cell wall skeleton (CWS) in a 2% squalene/Tween™ 80 emulsion. This adjuvant can be prepared by the methods taught in GB 2122204B. A preferred form of 3-de-O-acylated monophosphoryl lipid A is in the form of an emulsion having a small particle size of less than 0.2 μm in diameter (EP 0689454 B1).

The muramyl dipeptide adjuvants include N-acetyl-muramyl-L-threonyl-D-isoglutamine (thr-MDP; U.S. Pat. No. 4,606,918), N-acetyl-normuramyl-L-alanyl-D-isoglutamine (CGP 11637, referred to as nor-MDP), and N-acteryl-muramyl-L-alanyl-D-isoglutaminyl-L-alanine-2-(1′,2′-dipalmitoyl-sn-glycero-3-hydroxyphosphoryloxy)-ethylamin (CGP) 1983A, referred to as MTP-PE.

Desirable adjuvant mixtures include combinations of 3D-MPL and QS21 (EP0671948 B1), oil-in-water emulsions comprising 3D-MPL and QS21 (WO 95/17210, PCT/EP98/05714), 3D-MPL formulated with other carriers (EP 0689454 B1), QS21 formulated in cholesterol-containing liposomes (WO 96/33739), or immunostimulatory oligonucleotides (WO 96/02555). Alternative adjuvants include those described in WO 99/52549 and non-particulate suspensions of polyoxyethylene ether (UK Patent Application No. 9807805.8).

Adjuvants are utilized in an adjuvant amount, which can vary with the adjuvant, mammal, and the immunogenic CT144, CT242, and CT812 polypeptide, polypeptide fragment, or fusion protein. Typical amounts can vary from about 1 μg to about 1 mg per immunization. Those skilled in the art know that appropriate concentrations or amounts can be readily determined.

The present invention also provides DNA vaccines containing polynucleotide sequences encoding the polypeptides, polypeptide fragments, and fusion proteins of the present invention. Methods for the preparation of DNA vaccines which contain polynucleotide sequences encoding the CT144, CT242, or CT812 polypeptides, polypeptide fragments, or fusion proteins of the present invention are known in the art. For example, the polynucleotide sequences encoding the CT144, CT242, or CT812 polypeptides, polypeptide fragments, or fusion proteins of the present invention may be placed into virus-based vectors, which transfer the CT144, CT242, or CT812 polypeptide-, polypeptide fragment-, or fusion protein-encoding polynucleotide sequence (e.g., DNA or RNA) into a cell, such that the encoded polypeptide, polypeptide fragment, or fusion protein is expressed in the cell. Different viral-based vectors that may be used to deliver the CT144, CT242, or CT812 polypeptide-, polypeptide fragment-, or fusion protein-encoding polynucleotide sequences include adenoviral vectors and adeno-associated virus-derived vectors, retroviral vectors, Moloney Murine Leukemia virus-based vectors, Spleen Necrosis Virus-based vectors, Friend Murine Leukemia-based vectors, lentivirus-based vectors (Lois et al., Science, 295:868-872, 2002), papova virus-based vectors (e.g., SV40 viral vectors), Herpes Virus-based vectors, viral vectors that contain or display the Vesicular Stomatitis Virus G-glycoprotein Spike, Semliki-Forest virus-based vectors, Hepadnavirus-based vectors, and Baculovirus-based vectors. Additional, exemplary DNA vaccine vectors (not intended as limiting) may be found in “Gene Transfer and Expression in Mammalian Cells,” Savvas C. Makrides (Ed.), Elsevier Science Ltd, 2003. The DNA vaccine may be provided to a subject in combination with one or more acceptable diluent vehicles, pharmaceutically acceptable carriers, adjuvants, excipients, wetting or emulsifying agents, or pH buffering agents (examples provided herein) and/or one or more nucleic acid delivery agents (e.g., polymer, lipid, peptide based, degradable particles, microemulsions, VPLs, attenuated bacterial or viral vectors) using any route of administration or ex vivo loading.

Vaccines are conventionally administered parenterally, by injection, for example, either subcutaneously or intramuscularly. Typically vaccines are prepared in an injectable form, either as a liquid solution or a suspension. Solid forms suitable for injection may also be prepared as emulsions, or with the immunogenic polypeptide, polypeptide fragment, or fusion protein encapsulated in liposomes. Additional formulations that are suitable for other modes of administration include suppositories and, in some cases, oral formulation or by nasal spray. For suppositories, traditional binders and carriers can include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like.

The vaccine composition takes the form of a solution, suspension, tablet, pill, capsule, sustained release formulation or powder, and contains an immunogenic effective amount of the disclosed CT144, CT242, and CT812 polypeptide, polypeptide fragment, fusion protein, or DNA vaccines. In a typical composition, an immunogenic effective amount of the immunogenic polypeptide, polypeptide fragment, fusion protein, or DNA vaccine is about 1 μg to 10 mg per dose, and more desirably, about 5 μg to 5 mg per dose.

A vaccine is typically formulated for parenteral administration. Exemplary immunizations are carried out sub-cutaneously (SC), intramuscularly (IM), intravenously (IV), intraperitoneally (IP), or intra-dermally (ID).

The immunogenic CT144, CT242, or CT812 polypeptides, polypeptide fragments, and fusion proteins described herein can be formulated into the vaccine as neutral or salt forms. Pharmaceutically acceptable salts, include the acid addition salts (formed with the free amino groups of the polypeptide, polypeptide fragment, or fusion protein) and are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as are therapeutically effective and immunogenic. The quantity to be administered depends on the subject to be treated, capacity of the subject's immune system to host an immune response, and the degree of protection desired (e.g., prophylactic treatment or treatment of a patient with Chlamydia). The precise amount of CT144, CT242, and CT812 polypeptide, polypeptide fragment, fusion protein, or DNA vaccine required to be administered depends on the judgment of the practitioner and are peculiar to each subject. However, suitable dosage ranges are of the order of several hundred of micrograms active ingredient per subject. Suitable regimes for initial administration and booster shots are also variable, but are typified by an initial administration followed in intervals (weeks or months) by a subsequent injection or other administration.

### Pharmaceutical Compositions

In addition to vaccines, the invention also provides pharmaceutical compositions that include CT144, CT242, or CT812 polypeptides, polypeptide fragments, or fusion proteins of the present invention. Such compositions may be incorporated into a pharmaceutically acceptable carrier, vehicle, or diluent.

In one embodiment, the pharmaceutical composition includes a pharmaceutically acceptable excipient. The compounds of the present invention may be administered by any suitable means, depending for example, on their intended use, as is well known in the art, based on the present description. For example, if the polypeptides, polypeptide fragments, or fusion proteins of the present invention are to be administered orally, they may be formulated as tablets, capsules, granules, powders, or syrups. Alternatively, formulations of the present invention may be administered parenterally as injections (intravenous, intramuscular, or subcutaneous), drop infusion preparations, or suppositories. For application by the ophthalmic mucous membrane route, the compounds of the present invention may be formulated as eye drops or eye ointments. Aqueous solutions are generally preferred for ocular administration, based on ease of formulation, biological compatibility, as well as a subject's ability to easily administer such compositions, for example, by means of instilling one to two drops of the solutions in the eye. However, the compositions may also be suspensions, viscous or semi-viscous gels, or other types of solid or semi-solid compositions.

The above-described formulations may be prepared by conventional means, and, if desired, the compounds may be mixed with any conventional additive, such as an excipient, a binder, a disintegrating agent, a lubricant, a corrigent, a solubilizing agent, a suspension aid, an emulsifying agent, or a coating agent.

Subject compounds may be suitable for oral, nasal, topical (including buccal and sublingual), rectal, vaginal, aerosol, and/or parenteral administration. The formulations may conveniently be presented in unit dosage form and may be prepared by any methods well known in the art of pharmacy. The amount of agent that may be combined with a carrier material to produce a single dose varies depending upon the subject being treated, and the particular mode of administration.

Pharmaceutical compositions of this invention suitable for parenteral administration includes one or more components of a supplement in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

### Methods of Treating Bacterial Infections

The polypeptide fragments, fusion proteins, pharmaceutical compositions, and vaccines described herein may be used in a variety of treatments of diseases including a bacterial infection, most preferably a C. trachomatis infection in a subject. Those skilled in the art will understand, the dosage of any composition described herein will vary depending, for example, on the symptoms, age, and body weight of the subject, the nature and severity of the infection to be treated or prevented, the route of administration, and the form of the supplement. Any of the subject formulations may be administered in any suitable dose, such as, for example, in a single dose or in divided doses. Dosages for the compounds of the present invention, alone or together with any other compound of the present invention, or in combination with any compound deemed useful for the particular infection to be treated, may be readily determined by techniques known to those skilled in the art. Also, the present invention provides mixtures of more than one subject compound, as well as other therapeutic agents.

The combined use of several compounds of the present invention, or alternatively other therapeutic agents, may reduce the required dosage for any individual component because the onset and duration of effect of the different components may be complimentary. In such combined therapy, the different active agents may be delivered together or separately, and simultaneously or at different times within the day.

Different bacterial infections that may be treated or prevented with the present invention include: Chlamydia pneumoniae, Chlamydia psittaci, and Chlamydia trachomatis.

### Therapeutic Antibodies and T-cell Depletion

Alternatively, the immune response to Chlamydia, rather than the infection itself, may be responsible for symptoms that accompany infection, including sterility and pelvic inflammatory disease in a subject. In this case, it may be desirable to limit the immune response by a subset of CD4⁺ or CD8⁺ T cells within an infected subject. Antibodies which specifically recognize T cell clones targeted to the polypeptides, polypeptide fragments, or fusion proteins of the present invention, may therefore be useful in treating or preventing deleterious effects associated with Chlamydia infection. Methods for selective depletion of specific populations of T cells are described, for example, in Weinberg et al. (Nature Med. 2:183-189, 1966).

The following Example is meant to illustrate the invention and should not be construed as limiting.

### Example 1

### Determining whether a C. trachomatis Polypeptide, Polypeptide Fragment, or Fusion Protein is Immunogenic

### Methods

A library of cells or viruses containing polynucleotides encoding C. trachomatis polypeptides, polypeptide fragments, or fusion proteins may be screened to determine which of the polypeptides, polypeptide fragments, or fusion proteins encoded by the polynucleotides are immunogenic. This may be accomplished by contacting each member of the library with a second cell (e.g., a macrophage or antigen presenting cell) capable of endocytosing the cell of the C. trachomatis library, and displaying portions of the expressed polypeptide of the library on the surface of the second cell (see, e.g., U.S. Pat. No. 6,008,415). The second cell is then contacted with a C. trachomatis-specific T cell (e.g., a C. trachomatis-specific CD4⁺ or CD8⁺ T cell) from an organism previously infected with C. trachomatis. The second cell may also be fixed (e.g., using paraformaldehyde) prior to contacting with a C. trachomatis-specific T cell. A C. trachomatis-specific T cell capable of binding a presented portion of the C. trachomatis protein, will result in secretion of cytokines. Cytokine secretion (e.g., secretion of IFN-γ, IL-2, or TNF) may be assayed for as known in the art, for example, using an ELISA assay.

In particular, murine H2^{b} bone marrow-derived macrophages (BMMs) were seeded at a density of 1×10⁵ cells/well in 96-well plates. Fourteen to sixteen hours later, an aliquot of a frozen C. trachomatis library was thawed. The media was aspirated from the BMMs and replaced with a library aliquot and 60 μL of fresh RP-10 media. After 1 hour at 37° C., the BMMs were washed with PBS, 100 μL of RP-10 media added, and the cells incubated an additional hour at 37° C. The BMMs were then fixed with 1% paraformaldehyde for 15 minutes and washed extensively with PBS. BMM fixation was found to greatly reduce the background level of IFN-γ secretion by T cells. T cells (either C. trachomatis-specific CD4⁺ or CD8⁺ murine T cells; 1×10⁵) were added to each well in 200 μL of RP-10 media. Plates were incubated for 18-24 hours at 37° C. and the amount of IFN-γ in the supernatant of each well determined through the use of an IFN-γ ELISA assay (Endogen).

Another way to identify an antigenic peptide is to pulse the polypeptide, polypeptide fragment, or fusion protein onto macrophages and screen for their ability to activate C. trachomatis-specific CD4⁺ or CD8⁺ murine T cells (as described above). Peptides used in such assays can be synthesized using methods known in the art. A polypeptide, polypeptide fragment, or fusion protein that is capable of activating the C. trachomatis-specific CD4⁺ or CD8⁺ murine T cells is deemed immunogenic.

C. trachomatis-Specific CD8⁺Murine T cells

Pools of activated CD8⁺ murine T cells for use in the identification of immunogenic C. trachomatis polypeptides, polypeptide fragments, and fusion proteins may be obtained using methods known in the art. Typically, in screening for antigens to pathogenic organisms, CD8⁺ T cells are prepared from a mammal previously infected with the pathogenic organism. This preparation contains CD8⁺ T cells specific for antigens from the pathogen.

C. trachomatis-specific CD8⁺ T cells were harvested from mice as follows. A C57BL/6 mouse was injected intraperitoneally with 10⁷ infection-forming units of C. trachomatis. Fourteen days later the mouse was euthanized and the spleen was harvested. The spleen was mashed through a 70 μm screen to create a single cell solution of splenocytes. The CD8⁺ T cells were isolated from the splenocytes using anti-CD8 antibodies bound to MACS™ magnetic beads and separation protocols standard in the art (see, for e.g., MACS™ technology available from Miltenyi Biotec Inc., Auburn, Calif.). The isolated CD8⁺ cells were added to macrophages of the same haplotype (H2^{b}), which were infected with C. trachomatis 18 hours prior in a 24-well dish. Irradiated splenocytes from a naïve mouse (C57BL/6) were added as feeder cells in media containing IL-2. The cells were incubated for 10 days during which the C. trachomatis-specific CD8⁺ T cells were stimulated by the infected macrophages and replicated. On day 10, the CD8⁺ T cells were stimulated again using macrophages infected with C. trachomatis (18 hours prior), and irradiated splenocytes. This procedure was repeated until sufficient amounts of CD8⁺ T cells were present to screen the library.

CD8⁺ T cells may also be cloned from a human subject as described by, for example, Hassell et al. (Immunology 79: 513-519, 1993).

C. trachomatis-Specific Murine CD4⁺ T Cells

Activated CD4⁺ murine T cells for use in the identification of immunogenic C. trachomatis polypeptides, polypeptide fragments, and fusion proteins may be obtained using methods known in the art. Splenocytes from mice were isolated 21 days after infection with C. trachomatis serovar L2 and cultured with irradiated (2,000 rad) bone marrow-derived dendritic cells, UV-inactivated C. trachomatis serovar L2, and naïve syngeneic splenocytes in RP-10 (RPMI medium 1640 supplemented with 10% fetal calf serum, L-glutamine, HEPES, 50 μM 2-β-mercaptoethanol, 50 units/ml penicillin, and 50 μg/ml streptomycin) with a-methyl mannoside and 5% supernatant from Con A-stimulated rat spleen cells. CD8⁺ T cells were depleted from the culture by using Dynabeads Mouse CD8 (Invitrogen, Carlsbad, Calif.). The CD4⁺ T cells were restimulated every 7 days with C. trachomatis-pulsed bone marrow-derived dendritic cells. Once a C. trachomatis-specific CD4⁺ cell line was established, a CD4⁺ T cell clone was isolated by limiting dilution.

CD4⁺ T cells may also be cloned from a human subject as described by, for example, Hassell et al. (Immunology 79: 513-519, 1993).

### Results

Using the above techniques, amino acids 67-86 (SEQ ID NO: 2) and amino acids 77-96 (SEQ ID NO: 3) of CT144 were identified as CD4⁺ murine T cell epitopes; and amino acids 109-117 (SEQ ID NO: 5) and amino acids 112-120 (SEQ ID NO: 6) of CT242, and amino acids 103-111 (SEQ ID NO: 8) of CT812 were identified as CD8⁺ murine T cell epitopes (Table 1), as they elicited at least a 40-fold increase in IFN-γ production in T-lymphocytes compared to T-lymphocytes treated with a non-antigenic peptide in the same assay (e.g., a peptide which elicits the lowest measurable value of IFN-γ in the same assay). While the CD4⁺ and CD8⁺ antigens described in this Example where identified using mouse T-cells, in view of this discovery and using the techniques described herein, one skilled in the art could also identify the corresponding CD4⁺ and CD8⁺ human T-cell antigens.

TABLE 1

| | | IFN-γ Produced | |
|---|---|---|---|
| Antigen | Peptides | ng | −fold* |

| | | | | |
|---|---|---|---|---|
| CT144 | AQGKLIVTNPKSDISFGGRV | (SEQ ID NO: 2) | 37.0 (±1.8) | 925 |
| (CD4⁺ | KSDISFGGRVNLADNTVNYS | (SEQ ID NO: 3) | 1.6 (±0.4) | 40 |
| Ag) | NVTQDLTSSTAKLECTQDLI | | 1.4 (±0.3) | 35 |
| | AKLECTQDLIAQGKLIVTNP | | 1.4 (±0.3) | 35 |
| CT242 | YQILNQSNL | (SEQ ID NO: 5) | >230 | >5,750 |
| (CD8⁺ | LNQSNLKRM | (SEQ ID NO: 6) | 6.5 (±2.4) | 160 |
| Ag) | SNLKRMQKI | | 0.40 (±0.07) | 10 |
| CT812 | FSVTNPVVF | (SEQ ID NO: 8) | 2.6 (±0.3) | 65 |
| (CD8⁺ | AALYSTEDL | | 0.02 (±0.02) | 0.5 |
| Ag) | FQEKDADTL | | 0.09 (±0.03) | 2.0 |
| | QSVNELVYV | | 0.01 (±0.01) | 0.3 |
| | LEFASCSSL | | 0.06 (±0.01) | 1.5 |
| | FTSSNLDSP | | 0.04 (±0.01) | 1.0 |
| | SQAEGQYRL | | 0.03 (±0.3) | 0.8 |
| | GQSVNELVY | | 0.03 (±0.02) | 0.8 |
| | QAVLLLDQI | | ND | ND |

The values are the mean (± standard deviation) of duplicate samples. ND represents an epitope which was not detected in the assay. Asterisk indicates fold-increase of IFN-γ production in T-lymphocytes treated with the polypeptide fragments compared to untreated T-lymphocytes.

All patents, patent applications, patent application publications, and other cited references are hereby incorporated by reference to the same extent as if each independent patent, patent application, or publication was specifically and individually indicated to be incorporated by reference.

What is claimed is:

1. An isolated CT144 polypeptide comprising an amino acid sequence substantially identical to SEQ ID NO: 1, or fragment thereof, wherein said polypeptide or fragment elicits at least an 40-fold increase in interferon-γ production from a population of T-lymphocytes compared to the level of interferon-γ production elicited from a non-antigenic peptide in the same assay.

2. The polypeptide or fragment of claim 1, wherein said polypeptide or fragment, when administered to a mammal, elicits an immune response.

3. The polypeptide or fragment of claim 1, wherein said fragment elicits a CD4⁺ T-cell response.

4. The fragment of claim 1, wherein said fragment comprises the sequence of SEQ ID NO: 2 or 3, and at least one flanking amino acid.

5. The fragment of claim 4, wherein said fragment is fewer than 200 amino acids in length.

6.-9. (canceled)

10. The fragment of claim 1, wherein said fragment consists of the sequence of SEQ ID NO: 2 or 3.

11. The fragment of claim 10, wherein said fragment is truncated at the N- and/or C-terminus by one, two, three, four, five, or six amino acids.

12. The fragment of claim 4, wherein said fragment contains one or more conservative amino acid substitutions in the sequence of SEQ ID NO: 2 or 3.

13.-16. (canceled)

17. The fragment of claim 10, wherein said fragment contains one or more conservative amino acid substitutions.

18.-21. (canceled)

22. A pharmaceutical composition comprising the polypeptide or fragment of claim 1 in a pharmaceutically acceptable carrier.

23. A vaccine comprising:
a) the polypeptide or fragment of claim 1, and
b) a pharmaceutically acceptable carrier.

24. A method of treating or preventing a bacterial infection, said method comprising administering to a subject in need thereof, a therapeutically effective amount of the polypeptide or fragment of claim 1.

25. (canceled)

26. The method of claim 24, wherein said polypeptide or fragment is capable of generating an immune response in said subject or wherein said bacterial infection is Chlamydia infection.

27. (canceled)

28. The method of claim 26, wherein said subject has or is at risk for contracting Chlamydia.

29. An isolated fragment of a CT242 polypeptide, wherein said fragment comprises the sequence of SEQ ID NO: 5 or 6, and is fewer than 170 amino acids in length, and wherein said fragment elicits at least an 40-fold increase in interferon-γ production from a population of T-lymphocytes compared to the level of interferon-γ production elicited from a non-antigenic peptide in the same assay.

30. The fragment of claim 29, wherein said fragment, when administered to a mammal, elicits an immune response.

31. The fragment of claim 29, wherein said fragment elicits a CD8⁺ T-cell response.

32. The fragment of claim 29, wherein said fragment comprises the sequence of SEQ ID NO: 5 or 6, and at least one flanking amino acid.

33.-37. (canceled)

38. The fragment of claim 29, wherein said fragment consists of the sequence of SEQ ID NO: 5 or 6.

39. The fragment of claim 38, wherein said fragment is truncated at the N- and/or C-terminus by one or two amino acids.

40. The fragment of claim 32, wherein said fragment contains one or more conservative amino acid substitutions in the sequence of SEQ ID NO: 5 or 6.

41.-43. (canceled)

44. The fragment of claim 38, wherein said fragment contains one or more conservative amino acid substitutions.

45.-47. (canceled)

48. A pharmaceutical composition comprising the fragment of claim 29 in a pharmaceutically acceptable carrier.

49. A vaccine comprising:
a) the fragment of claim 29, and
b) a pharmaceutically acceptable carrier.

50. A method of treating or preventing a bacterial infection, said method comprising administering to a subject in need thereof, a therapeutically effective amount of the fragment of claim 29.

51. (canceled)

52. The method of claim 50, wherein said fragment is capable of generating an immune response in said subject or wherein said bacterial infection is Chlamydia infection.

53. (canceled)

54. The method of claim 52, wherein said subject has or is at risk for contracting Chlamydia.

55. An isolated fragment of a CT812 polypeptide, wherein said fragment comprises the sequence of SEQ ID NO: 8, and is fewer than 770 amino acids in length, and wherein said fragment elicits at least an 40-fold increase in interferon-γ production from a population of T-lymphocytes compared to the level of interferon-γ production elicited from a non-antigenic peptide in the same assay.

56. The fragment of claim 55, wherein said fragment, when administered to a mammal, elicits an immune response.

57. The fragment of claim 55, wherein said fragment elicits a CD8⁺ T-cell response.

58. The fragment of claim 55, wherein said fragment comprises the sequence of SEQ ID NO: 8, and at least one flanking amino acid.

59.-65. (canceled)

66. The fragment of claim 55, wherein said fragment consists of the sequence of SEQ ID NO: 8.

67. The fragment of claim 66, wherein said fragment is truncated at the N- and/or C-terminus by one or two amino acids.

68. The fragment of claim 58, wherein said fragment contains one or more conservative amino acid substitutions in the sequence of SEQ ID NO: 8.

69.-71. (canceled)

72. The fragment of claim 66, wherein said fragment contains one or more conservative amino acid substitutions.

73.-75. (canceled)

76. A pharmaceutical composition comprising the fragment of claim 55 in a pharmaceutically acceptable carrier.

77. A vaccine comprising:
a) the fragment of claim 55, and
b) a pharmaceutically acceptable carrier.

78. A method of treating or preventing a bacterial infection, said method comprising administering to a subject in need thereof, a therapeutically effective amount of the fragment of claim 55.

79. (canceled)

80. The method of claim 78, wherein said fragment is capable of generating an immune response in said subject or wherein said bacterial infection is Chlamydia infection.

81. (canceled)

82. The method of claim 80, wherein said subject has or is at risk for contracting Chlamydia.

83. An isolated fusion protein comprising:
a) the polypeptide or fragment of claim 1; and
b) a fusion partner.

84. The fusion protein of claim 83, wherein said fragment comprises the sequence of SEQ ID NO: 2 or 3, and at least one flanking amino acid.

85. The fusion protein of claim 83, wherein said fragment consists of the sequence of SEQ ID NO: 2 or 3.

86. A pharmaceutical composition comprising the fusion protein of claim 83 in a pharmaceutically acceptable carrier.

87. A vaccine comprising:
a) the fusion protein of claim 83, and
b) a pharmaceutically acceptable carrier.

88. An isolated fusion protein comprising:
a) the fragment of claim 29; and
b) a fusion partner.

89. The fusion protein of claim 88, wherein said fragment comprises the sequence of SEQ ID NO: 5 or 6, and at least one flanking amino acid.

90. The fusion protein of claim 88, wherein said fragment consists of the sequence of SEQ ID NO: 5 or 6.

91. A pharmaceutical composition comprising the fusion protein of claim 88 and a pharmaceutically acceptable carrier.

92. A vaccine comprising:
a) the fusion protein of claim 88, and
b) a pharmaceutically acceptable carrier.

93. An isolated fusion protein comprising:
a) the fragment of claim 55; and
b) a fusion partner.

94. The fusion protein of claim 93, wherein said fragment comprises the sequence of SEQ ID NO: 8, and at least one flanking amino acid.

95. The fusion protein of claim 93, wherein said fragment consists of the sequence of SEQ ID NO: 8.

96. A pharmaceutical composition comprising the fusion protein of claim 93 in a pharmaceutically acceptable carrier.

97. A vaccine comprising:
a) the fusion protein of claim 93, and
b) a pharmaceutically acceptable carrier.

98. A DNA vaccine comprising a polynucleotide sequence that encodes the polypeptide or fragment of claim 1.

99. A DNA vaccine comprising a polynucleotide sequence that encodes the fusion protein of claim 83.

100. A method of treating or preventing a bacterial infection, said method comprising administering to a subject in need thereof, a therapeutically effective amount of the DNA vaccine of claim 98.

101. (canceled)

102. The method of claim 100, wherein said DNA vaccine is capable of generating an immune response in said subject or wherein said bacterial infection is Chlamydia infection.

103. (canceled)

104. The method of claim 102, wherein said subject has or is at risk for contracting Chlamydia.

105. A DNA vaccine comprising a polynucleotide sequence that encodes the fragment of claim 29.

106. A DNA vaccine comprising a polynucleotide sequence that encodes the fusion protein of claim 88.

107. A method of treating or preventing a bacterial infection, said method comprising administering to a subject in need thereof, a therapeutically effective amount of the DNA vaccine of claim 105.

108. (canceled)

109. The method of claim 107, wherein said DNA vaccine is capable of generating an immune response in said subject or wherein said bacterial infection is Chlamydia infection.

110. (canceled)

111. The method of claim 109, wherein said subject has or is at risk for contracting Chlamydia.

112. A DNA vaccine comprising a polynucleotide sequence that encodes the fragment of claim 55.

113. A DNA vaccine comprising a polynucleotide sequence that encodes the fusion protein of claim 93.

114. A method of treating or preventing a bacterial infection, said method comprising administering to a subject in need thereof, a therapeutically effective amount of the DNA vaccine of claim 112.

115. (canceled)

116. The method of claim 114, wherein said DNA vaccine is capable of generating an immune response in said subject or wherein said bacterial infection is Chlamydia infection.

117. (canceled)

118. The method of claim 116, wherein said subject has or is at risk for contracting Chlamydia.

119. A method of treating or preventing a bacterial infection, said method comprising administering to a subject in need thereof, a therapeutically effective amount of the DNA vaccine of claim 99.

120. The method of claim 119, wherein said DNA vaccine is capable of generating an immune response in said subject or wherein said bacterial infection is Chlamydia infection.

121. The method of claim 120, wherein said subject has or is at risk for contracting Chlamydia.

122. A method of treating or preventing a bacterial infection, said method comprising administering to a subject in need thereof, a therapeutically effective amount of the DNA vaccine of claim 106.

123. The method of claim 122, wherein said DNA vaccine is capable of generating an immune response in said subject or wherein said bacterial infection is Chlamydia infection.

124. The method of claim 123, wherein said subject has or is at risk for contracting Chlamydia.

125. A method of treating or preventing a bacterial infection, said method comprising administering to a subject in need thereof, a therapeutically effective amount of the DNA vaccine of claim 113.

126. The method of claim 125, wherein said DNA vaccine is capable of generating an immune response in said subject or wherein said bacterial infection is Chlamydia infection.

127. The method of claim 126, wherein said subject has or is at risk for contracting Chlamydia.
